SEQUENCE LISTING (15 sequences)

SEQ ID NO: 1 - protein, 9 aa, Artificial Sequence
  Description of Artificial Sequence Synthetic peptide
APYGWAPYR

SEQ ID NO: 2 - protein, 8 aa, Artificial Sequence
  Description of Artificial Sequence Synthetic peptide
VPGYYSTA

SEQ ID NO: 3 - protein, 17 aa, Artificial Sequence
  Description of Artificial Sequence Synthetic peptide
VTGDGPYYQYWFYESIS

SEQ ID NO: 4 - protein, 10 aa, Artificial Sequence
  Description of Artificial Sequence Synthetic peptide
APAHRYDYYR

SEQ ID NO: 5 - protein, 12 aa, Artificial Sequence
  Description of Artificial Sequence Synthetic peptide
VPPYYGYWYGTA

SEQ ID NO: 6 - protein, 11 aa, Artificial Sequence
  Description of Artificial Sequence Synthetic peptide
VTHYGGQPYIS

SEQ ID NO: 7 - protein, 6 aa, Artificial Sequence
  Description of Artificial Sequence Synthetic 6xHis tag
HHHHHH

SEQ ID NO: 8 - DNA, 33 bp, Artificial Sequence
  Description of Artificial Sequence Synthetic primer
atatatatataagcttgccgtttctgatgttcc

SEQ ID NO: 9 - DNA, 35 bp, Artificial Sequence
  Description of Artificial Sequence Synthetic primer
atatatatgaattcggtacggtagttaatcgagat

SEQ ID NO: 10 - protein, 10 aa, Unknown
  Description of Unknown Fibronectin type III sequence
APIKRRLYYR

SEQ ID NO: 11 - protein, 8 aa, Unknown
  Description of Unknown Fibronectin type III sequence
VPGSKSTA

SEQ ID NO: 12 - protein, 12 aa, Unknown
  Description of Unknown Fibronectin type III sequence
VTYPAPYSKPIS

SEQ ID NO: 13 - protein, 91 aa, Unknown
  Description of Unknown Fibronectin type III sequence
AVSDVPRKLEVVAATPTSLLISWDAPIKRRLYYRITYGETGGNSPVQEFTVPGSKSTATISGLKPGVDYTITVYAVTYPAPYSKPISINYR

SEQ ID NO: 14 - protein, 315 aa, Artificial Sequence
  Description of Artificial Sequence Synthetic polypeptide
METGLRWLLLVAVLKGVKLAVSDVPRKLEVVAATPTSLLISWDYGWAPYYRITYGETGGNSPVQEFTVPGYYSTATISGLKPGVDYTITVYAVTGDGPYYQYWFYESISINYRTEFPSTSSKPTSPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQR

SEQ ID NO: 15 - protein, 330 aa, Unknown
  Description of Unknown Fibronectin type III sequence
METGLRWLLLVAVLKGVKLAVSDVPRKLEVVAATPTSLLISWDAPIKRRLYYRITYGETGGNSPVQEFTVPGSKSTATISGLKPGVDYTITVYAVTYPAPYSKPISINYRTEFPSTSSKPTSPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYT

(19) United States
(12) Patent Application Publication
(10) Pub. No.: US 2020/0157187 A1
(43) Pub. Date: May 21, 2020

(54) ANTIGEN-SPECIFIC REAGENTS SPECIFIC TO ACTIVE TGF-BETA, METHODS OF PRODUCING THE SAME, AND METHODS OF USE

(71) Applicant: Loyola University Chicago, Maywood, IL (US)

(72) Inventor: Makio Iwashima, Oak Park, IL (US)

(21) Appl. No.: 16/655,454

(22) Filed: Oct. 17, 2019

Related U.S. Application Data
(60) Provisional application No. 62/747,085, filed on Oct. 17, 2018.

(51) Int. Cl. C07K 14/78; C07K 16/22; C07K 16/00

(52) U.S. Cl. CPC C07K 14/78; C07K 16/22; C07K 2317/526; C07K 2319/30; C07K 2317/524; C07K 16/005

(57) ABSTRACT

Antigen-specific reagents (antibody mimetics) that are artificial/mutated proteins and specifically detect active TGF-β. Also provided are methods for producing soluble forms of such antigen-specific reagents (and other affinity reagents), and methods of using the antigen-specific reagents, such as in imaging and flow cytometric analysis.

### CROSS-REFERENCE TO RELATED APPLICATIONS

This application claims the benefit of U.S. Provisional Application No. 62/747,085, filed Oct. 17, 2018, the contents of which are incorporated herein by reference.

### STATEMENT REGARDING FEDERALLY SPONSORED RESEARCH

This invention was made with government support under Grant No. 1R01AI100135-03 awarded by the U.S. National Institutes of Health (NIH) National Institutes of Allergy and Infectious Diseases (NIAID). The government has certain rights in the invention.

### SEQUENCE LISTING

The instant application contains a Sequence Listing which has been filed electronically in ASCII format and is hereby incorporated by reference in its entirety. Said ASCII copy, created on Jan. 31, 2020, is named B9-5660_SL.txt and is 9,817 bytes in size.

### BACKGROUND OF THE INVENTION

The present invention generally relates to transforming growth factor beta (TGF-β). The invention particularly relates to reagents that specifically detect active TGF-β in imaging and flow cytometric analysis, for example, as an approach for developing affinity reagents including inhibitors directed at TGF-β.

TGF-β is a pleiotropic cytokine that shapes differentiation of a wide range of cells including lymphocytes. Mammals express three types of TGF-β, namely TGF-β1, -β2, and -β3. All three forms of TGF-β are initially expressed as latent forms and require activation prior to binding to their common receptor, a heterodimer of TGF-βRI and RII. A majority of TGF-β present in the tissues is in the latent form.

Ubiquitously expressed in normal tissues and cell lines, TGF-β is involved in cell division, differentiation, cell motility, and cell death. For example, TGF-β causes growth and/or death of cancer cells and is a potent activator of epithelial-mesenchymal transformation (EMT), the process whereby epithelial cells acquire mesenchymal, fibroblast-like properties, and contributes to fibrosis and cancer metastasis. TGF-β modulates both myeloid and lymphoid cell functions and plays a critical role in the generation of effector and regulatory T cell subsets.

TGF-β family proteins are exported to the extracellular milieu in a latent form which consists of a dimer of the growth factor domain and a dimer of an inhibitory domain named Latency Associated Peptide (LAP). LAP is initially produced as a part of TGF-β protein and is cleaved from the growth factor domain and reassembled with the growth factor domain in a noncovalent manner. LAP-TGF-β complex (called Small Latency Complex: SLC) then associates with Latent TGF-β Binding Protein (LTBP) to form a large latency complex (LLC). LLC is exported to the extracellular environment and binds the extracellular matrix whereby TGF-β can be activated by various proteolytic or physical manipulations.

Compared to the process to generate membrane-bound LLC, the process involving the activation of TGF-β is not well characterized. Previous studies determined that Foxp3+ regulatory T cells (Tregs) express surface LAP and suppress other T cell activation in a TGF-β dependent manner. However, to date, there does not appear to exist any direct evidence regarding the potential expression of the active form of TGF-β on the surface of human Treg cells. A lack of reagents that specifically detect active TGF-β in imaging and flow cytometric analysis has limited an understanding of where and when active TGF-β is produced.

In view of the above, it would be desirable if it were possible to detect active TGF-β, as examples, for use in imaging and flow cytometric analysis.

### BRIEF DESCRIPTION OF THE INVENTION

The present invention provides antigen-specific reagents, also referred to herein as antibody mimetics, that are artificial/mutated proteins and specifically detect active TGF-β. The present invention further provides methods suitable for producing soluble forms of such antigen-specific reagents (and other affinity reagents), and methods of using the antigen-specific reagents, including but not limited to imaging and flow cytometric analysis.

According to one aspect of the invention, an antigen-specific reagent is provided that is a protein mutated to bind to active TGF-β but does not bind to latent TGF-β.

Another aspect of the invention is where the antigen specific reagent is a protein mutated from fibronectin type III domain (FN3) having an amino acid sequence modified at the BC loop as APYGWAPYR (SEQ ID NO: 1), at the DE loop as VPGYYSTA (SEQ ID NO: 2), and at the FG loop as VTGDGPYYQYWFYESIS (SEQ ID NO: 3).

Another aspect of the invention is where the antigen-specific reagent is a protein mutated from fibronectin type III domain (FN3) having an amino acid sequence modified at the BC loop as APAHRYDYYR (SEQ ID NO: 4), at the DE loop as VPPYYGYWYGTA (SEQ ID NO: 5), and at the FG loop as VTHYGGQPYIS (SEQ ID NO: 6).

According to another aspect of the invention, a method is provided that involves producing an antigen-specific reagent that is a mutated protein and specifically detects active TGF-β, and using the antigen-specific reagent for imaging and flow cytometric analysis to characterize TGF-β.

According to yet another aspect of the invention, a method is provided for producing a soluble affinity reagent that entails identifying a protein having a binding domain specific to an antigen, and fusing the binding domain of the protein with a constant region (Fc) of an immunoglobulin heavy or light chain to form the soluble affinity reagent.

Technical aspects of antigen-specific reagents as described above include their ability to detect active TGF-β and the ability to use the antigen-specific reagents in imaging and flow cytometric analysis for improved characterization of TGF-β. Another technical aspect is the ability to produce soluble forms of the antigen-specific reagents.

Other aspects and advantages of this invention will be appreciated from the following detailed description.

### BRIEF DESCRIPTION OF THE DRAWINGS

FIGS. 1A-1C represent active TGF-β binding clones isolated from a FN3 based phage display library. FIG. 1A displays data representing binding of phage clones to active TGF-β (black bars) or inactive TGF-β (open bars) detected by ELISA. Each phage clone was detected by anti-P8phage protein antibody. The x-axis shows the number of each clone. FIG. 1B discloses SEQ ID NOS 10-12 and 1-6, respectively, in order of appearance. FIG. 1C represents the sequence of wild type fibronectin highlighting the BC, DE and FG loops. FIG. 1C discloses SEQ ID NO: 13.

FIGS. 2A-2C represent surface expression of active TGF-β by human CD4⁺CD25⁺ Tregs. FIG. 2A represents flow cytometry analysis of human Tregs stained with phage directly conjugated to AF488. Human PBMC CD4⁺CD25⁺ Tregs were expanded in vitro, then stained with phage particles directly conjugated with Alexa Fluor 488. Cells were stained 1 hr at +4° C. with the TGF-β binding clones (#6 and #24 are shown) (solid line). Phage particles from the unfocused library were used as a negative control (dashed line). FIG. 2B represents inhibition of phage binding by TGF-β. Purified active TGF-β1 was added to the staining mixture containing clones 6 or 24. The mixture was used for Tregs staining prepared as in FIG. 2A. FIG. 2C represents confocal microscopy of Tregs stained with AF488-labeled phage particles (green), anti-CD25-AF647 (pseudo-red), and Hoechst-33342 (blue). LBR indicates phage particles obtained from the unfocused library. Images were collected using an LSM-510 confocal microscope with objective ×40.

FIGS. 3A-3C represent inhibition TGF-β signaling by affinity selected phage clones. FIG. 3A displays data related to inhibition of TGF-β-induced SMAD2/3 phosphorylation by phage particles. TGF-β1 (10 ng/ml) alone or TGF-β1 pre-incubated with phage particles (1 hour at RT) was added to Jurkat cells overnight. Phage particles from the unselected library were used as a negative control (LBR). Phosphorylation of SMAD2,3 was determined by flow cytometry. Solid lines show staining with the anti-phospho SMAD2,3 antibodies and dotted lines show isotype control staining. FIGS. 3B and 3C represent an effect of active TGF-β binding phages on epithelial mesenchymal transition NMuMG cells were stimulated for 48 hours by TGF-β (10 ng/ml) which was prepared with or without pre-incubation with a 1 OD concentration of phages (clone 6, 24, or unselected library: LBR as indicated). Cells were then analyzed by (B) light microscopy, or by (C) Western blot analysis of E-Cadherin. GAPDH was used as a loading amount control.

FIGS. 4A-4E represent purification of the active TGF-β binding protein. FIG. 4A is a schematic representation of the expression construct for a fibronectin-based monobody fusion protein with the rabbit IgG constant region. Leader: a rabbit VH leader peptide, FN3: target binding FN3 domain, Cγ2-Cγ3: CH2/CH3 domains of rabbit IgG, His6: (His) ×6 tag (SEQ ID NO: 7) attached to the C-terminus of the fusion protein. FIG. 4B represents protein expression and purification of 6Fc. The 6Fc protein was expressed by stably transfected CHO cells. From the culture supernatant, the fusion protein was purified by protein A column, and analyzed by SDS-PAGE (left), and by Western blot (right). The arrows indicate the bands correspond to the expected molecular weight (about 40 kDa) of the fusion protein. FIG. 4C represents TGF-beta binding of 6Fc detected by ELISA. Binding of 6Fc (top) or WTFc (bottom) to active TGF-β (closed bars) or LAP (open bars) was determined by ELISA. Assay plates were coated with each ligand (30 ng/well), and binding affinity of each protein was estimated by serial dilution as indicated on X-axis. FIG. 4D represents binding specificity of 6Fc to TGF-β isoforms. 6Fc affinities toward TGF-β1 (black bars), -β2 (gray bars) and -β3 (open bars) were compared by ELISA. Assay plates were coated with each ligand (30 μg/well), and a serially diluted 6Fc solution was added to each well. The striped bar shows background (no 6Fc added). WTFc did not bind any targets at the tested dilutions (not shown). FIG. 4E represents surface plasmon resonance analysis of 6Fc. Sensorgram of 6Fc protein was determined using different concentrations of 6Fc (shown on the right) on the chip coated with 100 RU of active TGF-β.

FIGS. 5A and 5B represent functional characterization of 6Fc. FIG. 5A represents an effect of 6Fc on phosphorylation of SMAD2/3. Jurkat cells were incubated overnight with TGF-β1 (10 ng/ml) pre-incubated with or without 6FC (125 or 50 μg/ml) 1 hour, at RT. As a control, WTFc was used (shown on the right panel). Phosphorylation of SMAD2,3 was assessed by flow cytometry (solid line). Dotted lines show isotype control staining. FIG. 5B represents confocal images of Tregs stained with the monobody-Ig fusion proteins. Purified human PBMC-derived Tregs were mixed with 6Fc or WTFc (40 μg/ml), followed by anti-rabbit Fab-AF488 (green) and Hoechst-33342 (blue) staining. Single-cell images were obtained using an LSM-510 confocal microscope (×40).

FIG. 6 represents the expression construct for a fibronectin-based monobody fusion protein with the rabbit IgG constant region. FIG. 6 discloses SEQ ID NOS 7 and 14-15, respectively, in order of appearance.

FIG. 7 contains Table 1, which contains data relating to the kinetic analysis of monobodies and 1D11 antibody.

### DETAILED DESCRIPTION OF THE INVENTION

Disclosed herein are antigen-specific reagents (e.g., antibody mimetics) that are created by mutating proteins to be able to specifically detect active TGF-β, thereby enabling the reagents to be useful in imaging and flow cytometric analysis. Also disclosed are methods for producing such antigen-specific reagents, and particularly methods for producing soluble forms of these reagents and other affinity reagents.

As discussed in more detail below in reference to investigation leading to the present invention, specific reagents that recognize active TGF-β were identified using a fibronectin-based phage display library and phage clones that bind the active form of TGF-β, but not to the latent form of TGF-β, were engineered and isolated. These clones can detect active TGF-β in immunofluorescent analysis.

One of the technical hurdles for phage display technology is solubilization of the antigen binding component. When expressed in E. coli as a truncated protein, all clones identified in the below investigations became insoluble. To overcome this problem, the antigen-binding domain was fused with a constant region (Fc) of an immunoglobulin heavy chain. In the nonlimiting investigations described below, a portion of the rabbit IgG Fc region (CH2 and CD3 domains) was utilized as the constant region (Fc) of an immunoglobulin heavy chain to which the antigen-binding domain was fused. This fusion protein was successfully produced by CHO cells and was easily purified by affinity chromatography. These fusion proteins, termed monobody-Fc, identified the presence of active TGF-β on the cell surface of human Tregs, providing fresh insight into Treg biology and suggesting a new approach for developing TGF-β inhibitors. The solubilized proteins did not lose the original antigen specificity suggesting that post-translational modifications or fusion with IgG did not affect the folding of the antigen binding domain.

As TGF-β contributes to fibrosis and cancer development, this phage display-derived/Ig fusion technique provides a path to the development of a variety of affinity reagents, including inhibitors directed at TGF-β and other targets of interest.

Nonlimiting embodiments of the invention will now be described in reference to experimental investigations leading up to the invention.

Tests were initially performed to identify and isolate active TGF-β specific monobodies. To isolate affinity reagents that bind active TGF-β1, a phage display library based on the fibronectin type III domain (FN3) was used. FN3 is a small (94 amino acids), Ig-like protein, and thermally stable. From FN3-based libraries, highly selective affinity reagents, termed “monobodies” were obtained against a variety of targets including Fyn tyrosine kinase, Src kinase, tumor necrosis factor, ubiquitin, estrogen receptor, and integrin.

FN3 does not contain disulfide bonds and is particularly suitable for probing intracellular targets. FN3 contains three loops that mirror complementarity determining regions (CDRs) of immunoglobulin. A library of proteins mutated at these three regions was generated. Specifically, a phage-display library of FN3 monobodies was constructed by randomization of three binding loops, BC, DE and FG. Mutagenesis was performed using primer extension mutagenesis powered by selective rolling circle amplification, Kunkel mutagenesis, and rolling circle amplification. The oligonucleotide primers were synthesized with mixtures of triplet phosphoramidite nucleotides (Ella biotech, Munich, Germany). For BC and FG loops, each randomized triplet of the oligonucleotides encoded 18 amino acid residues, consisting of 30% tyrosine, 15% serine, 15% glycine, 5% of tryptophan or phenylalanine, and 2.3% each of all other residues except cysteine and methionine. For DE loop each triplet encoded only four residues, which are 40% tyrosine, 25% serine, 25% glycine and 10% tryptophan. The oligonucleotide length for three loops was 5-8 residues (BC loop); 3,4,6-8 (DE loop); 7, 9-13 (FG loop). The covalently closed circular DNA was generated by Kunkel mutagenesis. DNA treated with uracil-DNA glycosylase (New England Biolabs, Ipswich, Mass.) for 2 hours was used as the template for rolling circle amplification. Synthesized double-stranded DNA was digested with NotI and religated with T4 DNA ligase (New England Biolabs, Ipswich, Mass.), purified with PCR cleanup kit (Qiagen; Valencia, Calif.), and total 122 μg DNA was electroporated into TG1 cells (Lucigen; Madison, Wis.). The estimated transformant number was 1.3×10¹¹, and final diversity of the library was calculated to be 1.1×10¹¹ as 84% of the transformants had all three loops mutated based on DNA sequences analysis.

To prepare phage particles displaying the recombinant FN3 monobodies, bacteria were grown at 37° C., 250 rpm in 2XYT media (Fisher, USA) with 50 μg/ml carbenicillin to density OD₆₀₀ₙₘ=0.5, infected with M13K07 helper phage (MOI=10) (NEB, MA), incubated 1 hour at 37° C., 150 rpm; TG1 cells resuspended in fresh 2XYT media with 50 μg/ml carbenicillin and 100 ug/ml kanamycin were cultured overnight at 30° C., 230 rpm. Phage particles were precipitated 1 hour at +4° C. from bacterial supernatant by 6% PEG 8000, 300 mM NaCl (final concentrations), centrifuged 12,000 rpm, 20 min, dissolved in phosphate buffered saline (PBS) and stored in 16% glycerol at −80° C. long term or at +4° C. for short-term storage.

To isolate active TGF-β specific clones, clones that bind recombinant full-length LAP protein conjugated beads (Met1-Ser390) were removed. Next, phages that did not bind to LAP beads were incubated with active-TGF-β (Ala279-Ser390)-attached beads. Specifically, to isolate monobodies that bind active TGF-β, two rounds of affinity selections were performed. Recombinant TGF-β1 and latent TGF-β1 (LAP associated) proteins (Sino Biological; China) were biotinylated with Lightning-Link biotin conjugation kit (Innova Biosciences; UK) and attached to streptavidin-magnetic beads (Promega; Madison, Wis.). During the first round, LAP-coated streptavidin-beads were incubated 2 hours at RT with library phage particles (negative selection). The beads were pulled down with a magnet, and unbound phage was incubated 2 hours at RT on active TGF-β conjugated beads (positive selection). After four washes, beads-bound phages were eluted with 100 mM glycine, pH 2.0, neutralized and infected into TG1 cells. Infected E. Coli was spread on agar plates containing 100 μg/ml carbenicillin, and grown at 30° C. overnight. The next day, colonies were scraped from plates and about 5×10⁸ cells were inoculated into 50 ml of 2XYT, 50 μg/ml carbenicillin and grown for 2-3 hours at 37° C., 250 rpm. When the culture reached an OD_{600 nm}=0.4-0.6, the cells were rescued with M13K07 helper phage (MOI=10) for 1 h, transferred to fresh 2XYT with 50 μg/ml carbenicillin, 100 μg/ml kanamycin, and grown overnight at 30° C. PEG-precipitated particles were used for the second round of selection.

In the second round, TGF-β bound streptavidin beads were incubated 4 hours at +4° C. with the phage in the presence of soluble LAP (unbiotinylated, 500 ng/ml). Beads pulled down with the magnet were washed 6 times in PBS with 400 mM KCL, eight times in PBS with 0.01% Tween-20, and phage particles were eluted with 100 mM glycine, pH 2.0, neutralized, infected into TG1 cells and plated. After this selection, phages from each colony were tested for their binding activity to active TGF-β by ELISA.

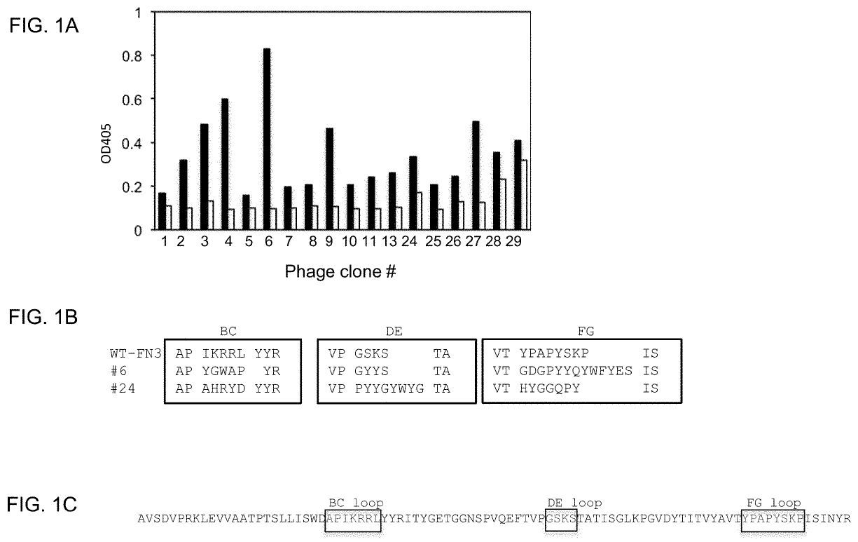

After two rounds of selection, 16 clones were obtained that bound active TGF-β with little to no cross-reactivity to LAP (FIG. 1A). These clones were non-redundant as they encoded unique amino acid sequences in each loop based on the DNA sequence (Representative clones are shown in FIG. 1B).

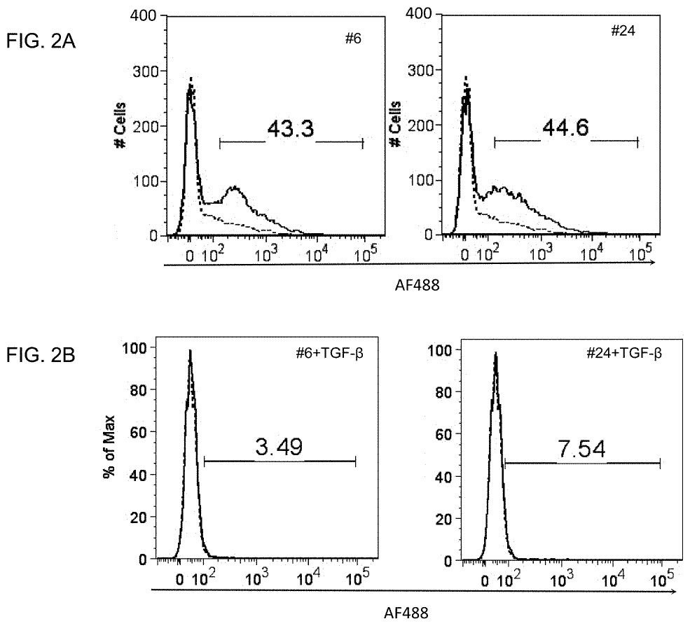

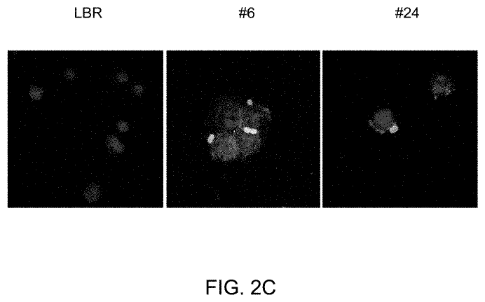

To develop an alternative approach to detection of TGF-β, tests were conducted to determine if isolated phage clones can detect active TGF-β by flow cytometry. Human CD4+CD25+ Tregs was used as the target as functional studies of human T regulatory cells (Tregs) demonstrated that these cells retain TGF-β on their cell surface. CD4+CD25+ Tregs were isolated from human PBMCs and expanded in vitro, then stained with phage particles directly conjugated to a fluorochrome (AF488) (FIG. 2A). Over 40% of cells stained with clone 6 or clone 24 were AF488 positive over the negative control. To confirm the specificity, these phage clones were also pre-incubated with active TGF-β protein before they were added to the cells (FIG. 2B). The percent positive cells stained with clone 6 and clone 24 was reduced from 43% to 3.5% and 44.6% to 7.5% respectively, demonstrating specificity.

To further examine the presence of active TGF-β on the regulatory T cell surface, the spatial distribution of active TGF-β was assessed by confocal microscopy (FIG. 2C). Cells stained with library phage (control) did not show significant AF488 signal, while phage clones 6 and 24 cells revealed the punctate distribution of fluorescence, indicating that active TGF-β localizes in a unique manner where proteins are anchored in a concentrated area of the T cell surface.

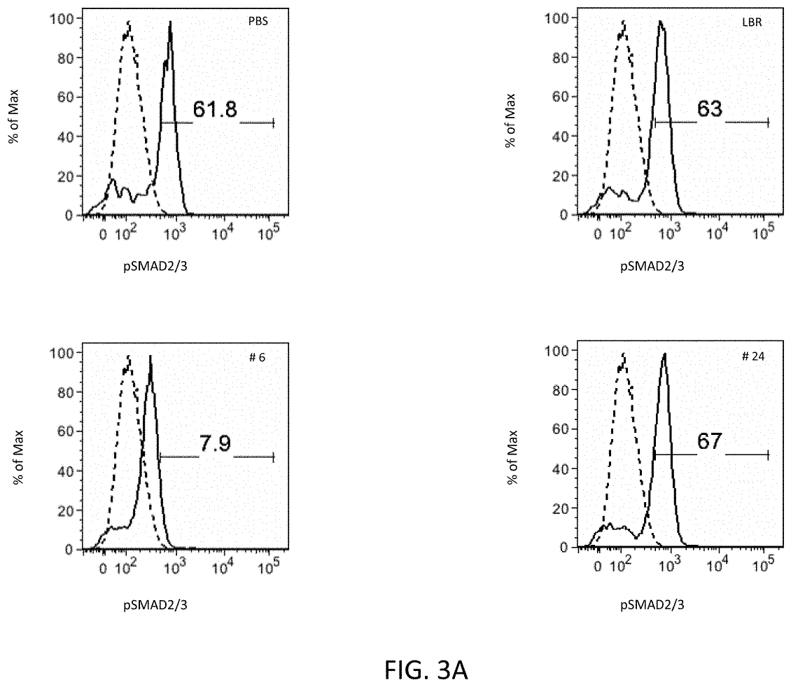

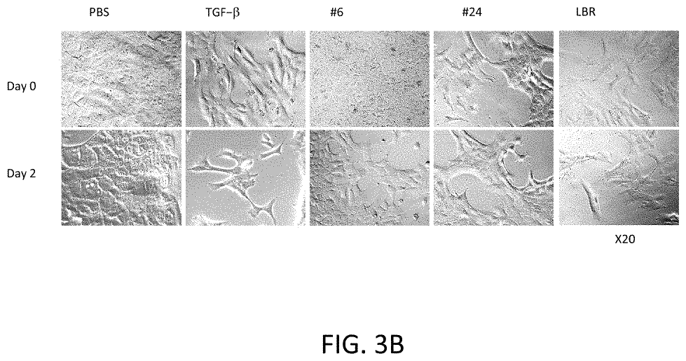

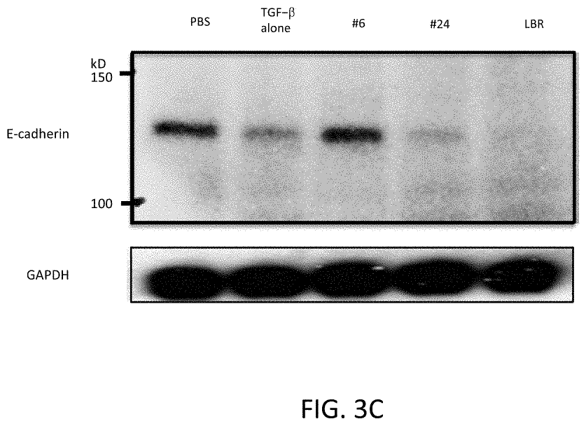

Next, tests were performed to determine if TGF-β binding monobodies inhibit TGF-β signaling by assessing the effect on TGF-induced downstream events. TGF-β was pre-incubated with phage particles from several TGF-β binding clones. The mixture was then added to Jurkat cells and cultured overnight. After the culture, cells were permeabilized and stained with the antibody against phosphorylated SMAD2,3 (FIG. 3A). While greater than 60% of cells incubated with TGF-β were pSMAD2/3 positive, pre-incubation of TGF-β with clone 6 reduced pSMAD2/3 positive cells to 7.9%. Other clones did not show significant changes, suggesting that only clone 6 could competitively inhibit active TGF-β binding to its receptor.

Based on the data shown above, it was hypothesized that clone 6 may inhibit biological process induced by TGF-β. To test this, the effect of clone 6 on epithelial-mesenchymal transformation (EMT) was examined. Prior studies suggest that exogenously added active TGFβ1 induces EMT in NMuMG cells. To test if clone 6 inhibits EMT, TGF-β1 was pre-incubated with purified phages, then added the mixture to NMuMG cells. After two days of culture, cells were imaged by light microscopy (FIG. 3B). NMUMG cells at Day 0 demonstrated a typical “honeycomb” morphology. The addition of active TGF-β induced a change of cell shape into fibroblast-like cells, suggesting that cells underwent EMT. When TGF-β was pre-incubated with phage clone 6, NMUMG cells kept the initial honeycomb morphology, suggesting inhibition of EMT by clone 6. Another clone 24 or wild-type phages did not show any inhibitory effect, demonstrating that clone 6-mediated inhibition of EMT is TGF-β binding-site specific.

Reduction of E-cadherin expression is a main molecular marker of EMT. If clone 6 blocks TGF-β-induced EMT, expression of E-cadherin would remain unchanged in cells that were cultured with clone 6. To test this, the expression of E-cadherin by NMuMG cells was determined after two days of TGF-β treatment (FIG. 3C). TGF-β alone, TGF-β pretreated with clone 24 or the total library phage, induced a significant reduction of E-cadherin expression. In contrast, cells treated with clone 6 did not show substantial changes in the E-cadherin level. Together with the outcomes of morphological analysis, it was concluded that clone 6 inhibits TGF-β to induce biological outcomes.

All further experiments were performed with clone 6 due to it having demonstrated the highest specificity and binding to active TGF-β. While monobodies displayed on the phage surface can be used as an affinity reagent, a soluble form of antigen binding protein is more robust both for in vitro and in vivo applications. FN3 is relatively stable, and it can be expressed as a soluble protein from bacterial expression systems. However, clone 6 was not soluble when expressed separately from the phage.

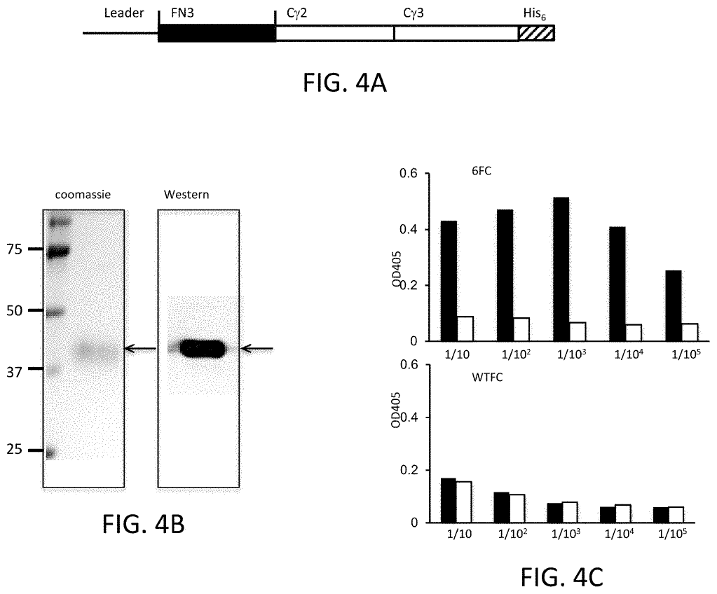

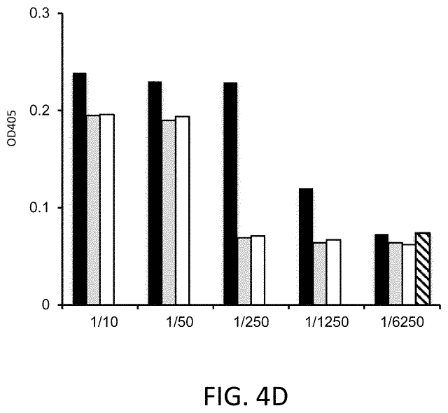

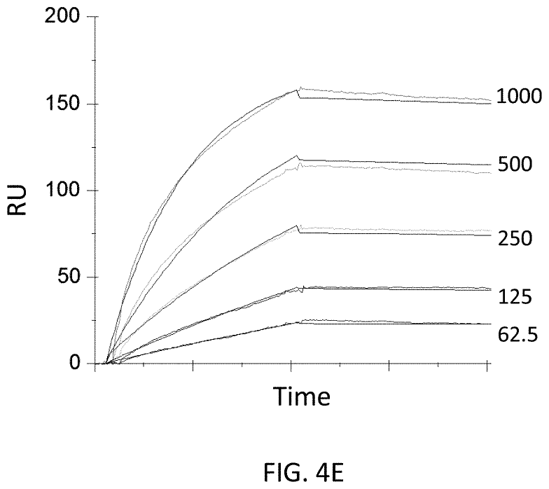

As an alternative to generating the soluble protein, a mammalian expression system was used to make an expression construct that fuses rabbit IgG constant region (CH2/CH3) to the antigen-binding domain of clone 6 (FIG. 4A). CHO cells were stably transfected with the expression construct, and the monobody-Fc fusion protein was purified using a Protein A column. Purified clone 6 fusion protein (termed 6Fc) demonstrated MW about 40 kDa with greater than 75% purity by SDS-PAGE (FIG. 4B, left), without any signs of degradation detected by Western blot (FIG. 4B, right). Similarly, the wild-type FN3 fusion (WTFc) protein was generated and purified.

The coding sequence of the wild-type or phage clone 6 FN3 monobody was PCR amplified with the following primers: Fw primer 5′-ATATATATATAAGCTTGCCGTTTCTGATGTTCC-3′ (SEQ ID NO: 8); Rv primer 5′-ATATATATGAATTCGGTACGGTAGTTAATCGAGAT-3′ (SEQ ID NO: 9). PCR products were cloned between the HindIII and EcoRI sites of the expression vector containing rabbit IgG construct region (Fc region). His6-tag (SEQ ID NO: 7) was fused to 3′ end of a Cγ3 domain (see FIG. 4A). Final constructs were sequence verified and designated WTFC (wild-type) and 6FC (clone 6). Chinese hamster ovary cells (CHO) were transfected with the expression vector for WTFC and 6FC using Lipofectamine-200 (Promega, Madison, Wis.), and stable transfectants secreting mono-Fc proteins were selected. Protein was single-step purified from supernatants using Protein A (GE Healthcare, UK) or Protein A followed by Immobilized Metal Affinity Chromatography (IMAC).

Protein purity was assessed by SDS-PAGE and Western blot; binding activity was determined by ELISA. Protein 6FC and WTFC were stored in PBS, 0.001% azide, 200 ug/ml BSA at +4° C. Antigen specificity of purified 6Fc and WTFc was tested by ELISA using active TGF-β and LAP as the antigens (FIG. 4C). 6Fc maintained specific binding to TGF-β (top panel) while a control WTFc did not bind either target (bottom panel). These studies suggest that 6Fc fusion protein was soluble and maintained its binding specificity.

TGF-β1 shares high amino acid homology with TGF-β2 and 3 (74%-78% respectively). Thus, tests were conducted to determine if 6Fc crossreacts with TGF-β2 and/or TGF-β3 by ELISA (FIG. 4D). 6FC bound to TGF-β1 at dilution 1/1250 of the applied samples while a similar level of binding was detected to TGF-β2 and -β3 at dilution 1/250. Of note, 6FC demonstrated greater than five fold higher binding ability to TGF-β1 than to TGF-β2 and 3.

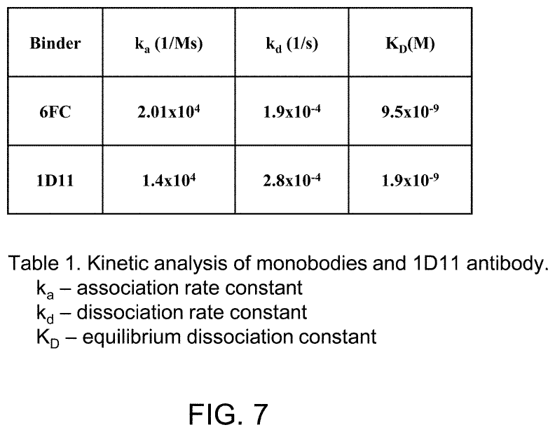

Binding kinetics and affinity for 6Fc was determined by surface plasmon resonance (SPR). An anti-TGF-βmonoclonal antibody 1 D11 was used as a positive control (FIG. 4E). 1D11 (fresolimumab) binds TGF-β1,2,3 and suppresses lung metastasis in metastatic breast cancer model. Fresolimumab has been used for a phase preclinical I trial in patients with malignant melanoma or renal cell carcinoma and showed no dose-limiting toxicity and preliminary evidence of antitumor activity. While K_{D} for recombinant monobody 6Fc was about five fold lower compared to 1D11 (9.5 nM and 1.9 nM respectively), the range of equilibrium association and dissociation rate constant was comparable (FIG. 7, Table 1). Based on these results, it was concluded that 6Fc mono-FC recognized active TGF-β at the sensitivity comparable to a pre-clinically utilized antibody.

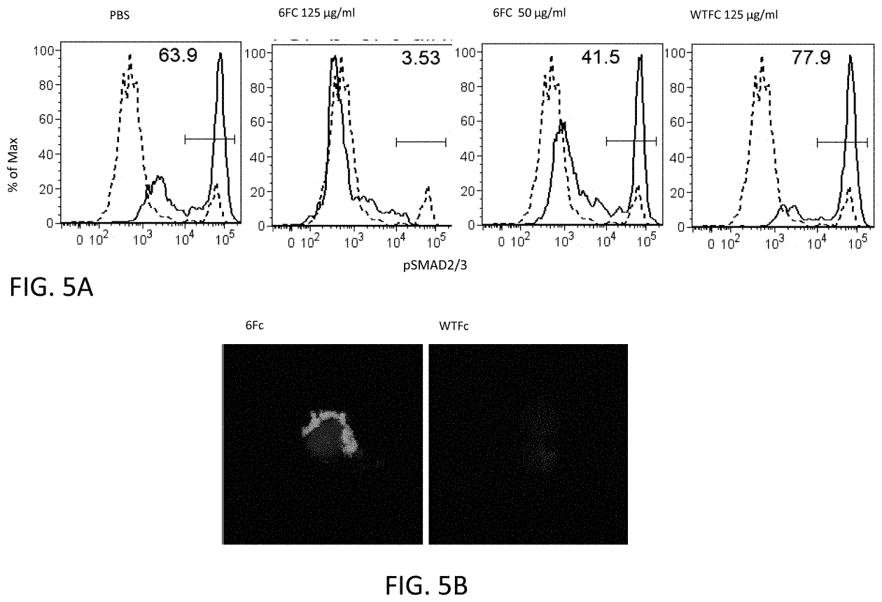

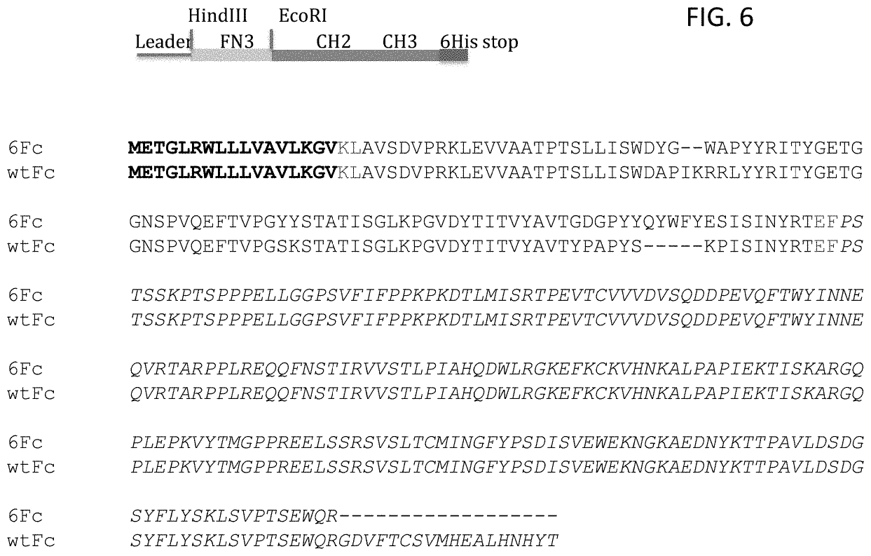

Tests were performed to determine if 6Fc inhibits TGF-β functions by determining signaling events downstream of the TGF-β receptor. A graded concentration of 6Fc (125, 50, 12.5 ug/ml) was pre-incubated with active TGF-β for one hour, then added the mixture to Jurkat cells. After overnight incubation, cells were permeabilized and stained with the antibody against phosphorylated SMAD2,3 (FIG. 5A). While WTFc showed no notable effect on activation of SMDA2/3, 6FC pre-incubation markedly reduced TGF-β-induced pSMAD2/3, demonstrating that 6Fc abrogates TGF-β binding to its receptor. Tests were also conducted to determine if 6Fc maintains the ability of clone 6 to detect surface TGF-β. In vitro expanded Tregs were stained with purified 6Fc or with WTFc. Confocal microscopy showed bright staining of Tregs by 6Fc but not by WTFc. Together, this data suggests that 6Fc maintained clone 6's specificity to block TGF-β to bind its receptor and to detect active TGF-β on the cell surface.

Though in the investigations, the antigen-binding domain was fused to the constant region of an immunoglobulin heavy chain, similar results are indicated for an immunoglobulin light chain. Immunoglobulin light chain constant regions (kappa and lambda chain) have a single domain that has high homology to an immunoglobulin heavy chain. It is known that immunoglobulin light chain can be secreted as homo dimer and tetramer as a natural product. It is believed that the antigen binding domain fused with the constant region of an immunoglobulin light chain would yield a stable soluble protein, and furthermore would be capable of reaching sites that cannot be accessed by an immunoglobulin heavy chain due to the latter's larger structure. Additionally, an immunoglobulin light chain does not bind Fc receptors expressed by various cells, and thus would have the advantage of being less toxic for the host and can have a longer half life in vivo.

While the invention has been described in terms of specific or particular embodiments and investigations, it should be apparent that alternatives could be adopted by one skilled in the art. For example, the specific antibody mimetics could differ in appearance and construction from the embodiments described herein, and process parameters such as temperatures and durations could be modified. Accordingly, it should be understood that the invention is not necessarily limited to any embodiment described herein. It should also be understood that the phraseology and terminology employed above are for the purpose of describing the disclosed embodiments and investigations, and do not necessarily serve as limitations to the scope of the invention. Therefore, the scope of the invention is to be limited only by the following claims.

1. An antigen specific reagent that is a mutated protein and specifically detects active TGF β.

2. The antigen-specific reagent of claim 1, wherein the antigen-specific reagent binds to active TGF β but does not bind to latent TGF-β.

3. The antigen-specific reagent of claim 1, wherein the antigen-specific reagent is soluble.

4. The antigen-specific reagent of claim 1, wherein the antigen-specific reagent is a protein mutated from fibronectin type III domain (FN3) having an amino acid sequence modified at the BC loop as APYGWAPYR (SEQ ID NO: 1), at the DE loop as VPGYYSTA (SEQ ID NO: 2), and at the FG loop as VTGDGPYYQYWFYESIS (SEQ ID NO: 3).

5. The antigen-specific reagent of claim 1, wherein the antigen-specific reagent is a protein mutated from fibronectin type III domain (FN3) having an amino acid sequence modified at the BC loop as APAHRYDYYR (SEQ ID NO: 4), at the DE loop as VPPYYGYWYGTA (SEQ ID NO: 5), and at the FG loop as VTHYGGQPYIS (SEQ ID NO: 6).

6. The antigen-specific reagent of claim 1, wherein the antigen-specific reagent is produced by a phage display process and is expressed on an exterior of a membrane.

7. The antigen-specific reagent of claim 1, wherein the antigen-specific reagent comprises a TGF-β binding domain fused with a constant region (Fc) of an immunoglobulin heavy or light chain.

8. The antigen-specific reagent of claim 1, wherein the antigen-specific reagent comprises a TGF-β binding domain fused with rabbit IgG constant region (CH2/CH3).

9. A method comprising:
producing an antigen-specific reagent that is a mutated protein and specifically detects active TGF β; and
using the antigen-specific reagent for imaging and flow cytometric analysis to characterize TGF β.

10. The method of claim 9, wherein the antigen-specific reagent binds to active TGF β but does not bind to latent TGF-β.

11. The method of claim 9, wherein the antigen-specific reagent is soluble.

12. The method of claim 9, wherein the antigen-specific reagent is a protein mutated from fibronectin type III domain (FN3) having an amino acid sequence modified at the BC loop as APYGWAPYR (SEQ ID NO: 1), at the DE loop as VPGYYSTA (SEQ ID NO: 2), and at the FG loop as VTGDGPYYQYWFYESIS (SEQ ID NO: 3).

13. The method of claim 9, wherein the antigen-specific reagent is a protein mutated from fibronectin type III domain (FN3) having an amino acid sequence modified at the BC loop as APAHRYDYYR (SEQ ID NO: 4), at the DE loop as VPPYYGYWYGTA (SEQ ID NO: 5), and at the FG loop as VTHYGGQPYIS (SEQ ID NO: 6).

14. The method of claim 9, wherein the antigen-specific reagent is produced by a phage display process and is expressed on an exterior of a membrane.

15. The method of claim 9, wherein the antigen-specific reagent comprises a TGF-β binding domain fused with a constant region (Fc) of an immunoglobulin heavy or light chain.

16. The method of claim 9, wherein the antigen-specific reagent comprises a TGF-β binding domain fused to rabbit IgG constant region (CH2/CH3).

17. A method for producing a soluble affinity reagent, the method comprising:
identifying a protein having a binding domain specific to an antigen; and
fusing the binding domain of the protein with a constant region (Fc) of an immunoglobulin heavy chain to form the soluble affinity reagent.

18. The method of claim 17, wherein the constant region (Fc) is rabbit IgG constant region (CH2/CH3).

19. The method of claim 17, wherein the soluble affinity reagent is an antigen-specific reagent.

20. The method of claim 19, wherein the antigen-specific reagent specifically detects active TGF β.
